(19) United States
(12) Patent Application Publication
(10) Pub. No.: US 2023/0172534 A1
(43) Pub. Date: Jun. 8, 2023

(54) DIGITAL TWIN MATCHING FOR THERAPEUTICS

(71) Applicant: AT&T Intellectual Property I, L.P., Atlanta, GA (US)

(72) Inventors: Brian M. Novack, St. Louis, MO (US); Rashmi Palamadai, Naperville, IL (US); Tan Xu, Bridgewater, NJ (US); Eric Zavesky, Austin, TX (US); Ari Craine, Marietta, GA (US); Richard Palazzo, Stewartsville, NJ (US); Robert Koch, Peachtree Corners, GA (US)

(21) Appl. No.: 17/541,090

(22) Filed: Dec. 2, 2021

(51) Int. Cl. A61B 5/00; A61B 5/024; A61B 5/0205; A61B 5/16

(52) U.S. Cl. CPC A61B 5/4848; A61B 5/02444; A61B 5/02055; A61B 5/4839; A61B 5/165

(57) ABSTRACT

The technology described herein is generally directed towards collecting digital twin datasets from individual users to represent their individual physical, emotional, chemical and/or environmental conditions. A user's digital twin is matched to one or more other digital twins with similar physical, emotional, chemical, and/or environmental conditions. The matched digital twins can share data and learnings via a virtual anonymous relationship. Multiple digital twins that represent users with similar conditions may be found and treated collectively as a group; a user via his or her digital twin can poll the group to receive and process responses from each respondent. A digital twin can be a therapist or a researcher who emulates a patient and uses the emulated digital twin as a proxy to monitor and process the results of other digital twins.

### TECHNICAL FIELD

The subject application relates to the collection of data related to a user's conditions for matching with data of similar users for anonymous sharing of information, and related embodiments.

### BACKGROUND

Medical conditions are numerous among any group of people. People often do not seek treatment for their conditions due to expense, fear, embarrassment or other similar reasons. A great deal of information is available to users of the Internet, but much of it may not be correctly located and applied. It would be beneficial if users could learn from other users like themselves who are experiencing similar physical, emotional, chemical, and/or environmental conditions.

### BRIEF DESCRIPTION OF THE DRAWINGS

Non-limiting and non-exhaustive embodiments of the subject disclosure are described with reference to the following figures, wherein like reference numerals refer to like parts throughout the various views unless otherwise specified.

FIG. 1 is a block diagram showing example components that facilitate creation of a digital twin of a user, in accordance with various aspects and embodiments of the subject disclosure.

FIG. 2 is an example representation of using on-board and external sensors in the creation of a digital twin of a user, in accordance with various aspects and embodiments of the subject disclosure.

FIG. 3 is an example representation of a user communicating with his or her digital twin, in accordance with various aspects and embodiments of the subject disclosure.

FIG. 4 is an example representation of a user receiving a report from his or her digital twin, in accordance with various aspects and embodiments of the subject disclosure.

FIG. 5 is an example representation of a chemical/drug delivery system coupled to a user for communicating with the user's digital twin, in accordance with various aspects and embodiments of the subject disclosure.

FIG. 6 is an example representation of a user with a digital twin locating a matching digital twin among a community of digital twins, and setting up a virtual anonymous relationship between them, in accordance with various aspects and embodiments of the subject disclosure

FIG. 7 is an example representation of a user with a digital twin locating a group of matching digital twins and setting up a virtual anonymous relationship between them, in accordance with various aspects and embodiments of the subject disclosure.

FIG. 8 is a flow diagram representing example operations related to storing digital twin data of users and finding matching digital twins, in accordance with various aspects and embodiments of the subject disclosure.

FIG. 9 is a flow diagram representing example operations related to finding a matching subgroup of digital twins, in accordance with various aspects and embodiments of the subject disclosure.

FIG. 10 is a flow diagram representing example operations related to collecting digital twin dataset of a group of users and using the datasets to match digital twins, in accordance with various aspects and embodiments of the subject disclosure.

FIG. 11 illustrates an example block diagram of an example mobile handset operable to engage in a system architecture that facilitates wireless communications according to one or more embodiments described herein.

FIG. 12 illustrates an example block diagram of an example computer/machine system operable to engage in a system architecture that facilitates wireless communications according to one or more embodiments described herein.

### DETAILED DESCRIPTION

The technology described herein is generally directed towards convenient and timely manner in which to find other users like themselves who are experiencing similar physical, emotional, chemical, and or environmental conditions and jointly monitor their progress in using a therapeutic to resolve or alleviate their problem. This disclosure describes a solution by enabling a user to create a data representation of a digital twin and to seek other similar digital twins to compare and learn from their results.

[symbol]

As used in this disclosure, in some embodiments, the terms “component,” “system” and the like are intended to refer to, or include, a computer-related entity or an entity related to an operational apparatus with one or more specific functionalities, wherein the entity can be either hardware, a combination of hardware and software, software, or software in execution. As an example, a component may be, but is not limited to being, a process running on a processor, a processor, an object, an executable, a thread of execution, computer-executable instructions, a program, and/or a computer. By way of illustration and not limitation, both an application running on a server and the server can be a component.

One or more components may reside within a process and/or thread of execution and a component may be localized on one computer and/or distributed between two or more computers. In addition, these components can execute from various computer readable media having various data structures stored thereon. The components may communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software application or firmware application executed by a processor, wherein the processor can be internal or external to the apparatus and executes at least a part of the software or firmware application. As yet another example, a component can be an apparatus that provides specific functionality through electronic components without mechanical parts, the electronic components can include a processor therein to execute software or firmware that confers at least in part the functionality of the electronic components. While various components have been illustrated as separate components, it will be appreciated that multiple components can be implemented as a single component, or a single component can be implemented as multiple components, without departing from example embodiments.

Further, the various embodiments can be implemented as a method, apparatus or article of manufacture using standard programming and/or engineering techniques to produce software, firmware, hardware or any combination thereof to control a computer to implement the disclosed subject matter. The term “article of manufacture” as used herein is intended to encompass a computer program accessible from any computer-readable (or machine-readable) device or computer-readable (or machine-readable) storage/communications media. For example, computer readable storage media can include, but are not limited to, magnetic storage devices (e.g., hard disk, floppy disk, magnetic strips), optical disks (e.g., compact disk (CD), digital versatile disk (DVD)), smart cards, and flash memory devices (e.g., card, stick, key drive). Of course, those skilled in the art will recognize many modifications can be made to this configuration without departing from the scope or spirit of the various embodiments.

Moreover, terms such as “mobile device equipment,” “mobile station,” “mobile,” subscriber station,” “access terminal,” “terminal,” “handset,” “communication device,” “mobile device” (and/or terms representing similar terminology) can refer to a wireless device utilized by a subscriber or mobile device of a wireless communication service to receive or convey data, control, voice, video, sound, gaming or substantially any data-stream or signaling-stream. The foregoing terms are utilized interchangeably herein and with reference to the related drawings. Likewise, the terms “access point (AP),” “Base Station (BS),” BS transceiver, BS device, cell site, cell site device, “gNode B (gNB),” “evolved Node B (eNode B),” “home Node B (HNB)” and the like, can be utilized interchangeably in the application, and can refer to a wireless network component or appliance that transmits and/or receives data, control, voice, video, sound, gaming or substantially any data-stream or signaling-stream from one or more subscriber stations. Data and signaling streams can be packetized or frame-based flows.

Furthermore, the terms “user equipment,” “device,” “communication device,” “mobile device,” “subscriber,” “customer entity,” “consumer,” “customer entity,” “entity” and the like may be employed interchangeably throughout, unless context warrants particular distinctions among the terms. It should be appreciated that such terms can refer to human entities or automated components supported through artificial intelligence (e.g., a capacity to make inference based on complex mathematical formalisms), which can provide simulated vision, sound recognition and so forth.

Embodiments described herein can be exploited in substantially any wireless communication technology, including, but not limited to, wireless fidelity (Wi-Fi), global system for mobile communications (GSM), universal mobile telecommunications system (UMTS), worldwide interoperability for microwave access (WiMAX), enhanced general packet radio service (enhanced GPRS), third generation partnership project (3GPP) long term evolution (LTE), third generation partnership project 2 (3GPP2) ultra mobile broadband (UMB), high speed packet access (HSPA), Z-Wave, Zigbee and other 802.11 wireless technologies and/or legacy telecommunication technologies.

One or more embodiments are now described with reference to the drawings, wherein like reference numerals are used to refer to like elements throughout. In the following description, for purposes of explanation, numerous specific details are set forth in order to provide a thorough understanding of the various embodiments. It is evident, however, that the various embodiments can be practiced without these specific details (and without applying to any particular networked environment or standard).

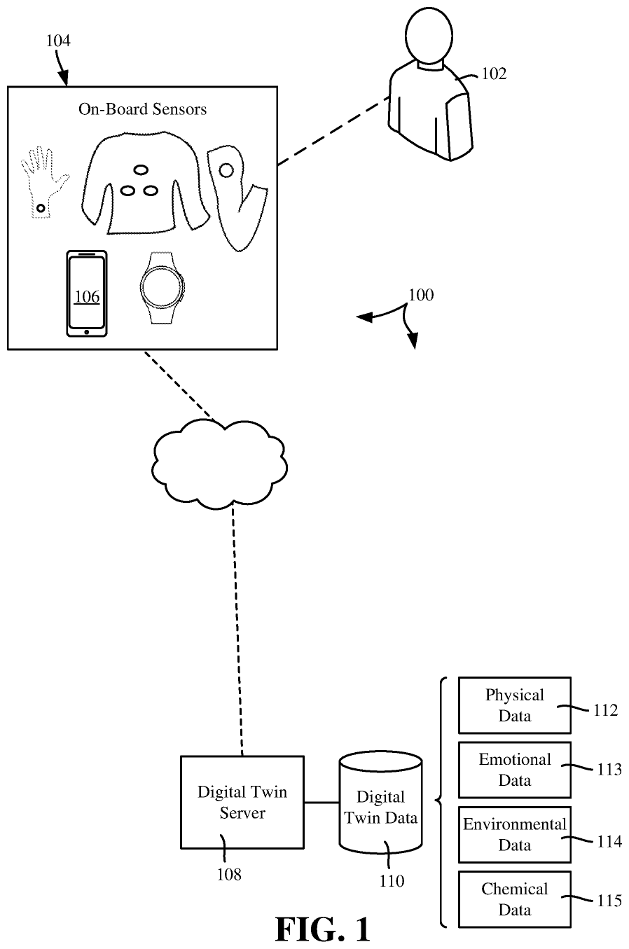

FIG. 1 is representation of example components of a system 100 that creates and maintains information of digital twins. In FIG. 1, a user is equipped with one or more on-board sensors 102 coupled to (or able to be held by) the user 102 in some suitable way. Such sensors include wearable, attached, hand-held and/or implanted sensors that may be used to monitor and collect data represents various aspects of the user's conditions. In FIG. 1, depicted on-board sensors can include, but are not limited to, at least one of wrist, torso (directly coupled or within a textile such as a shirt with embedded sensors) or arm sensors (represented as ovals or circles affixed to body portions/clothing depicted among the on-board sensors 104). Also shown is a fitness-type wristwatch that can detect data of the user such as heart rate, temperature, exercise, sleep and wake times and/or the like. A smartphone 106 can also capture some user data, as well as couple to other on-board sensors to communicate information from them to a digital twin server 108. It is understood that such on-board sensors are not limited to those depicted and/or described herein, nor are all of those depicted and/or described herein necessarily available or applicable to a given user.

The collected data may include the user's physical conditions, emotional conditions, chemical conditions, and environmental conditions. Physical data 112 may be detected using such sensors, such as heart rate sensors, respiratory sensors, medical tattoos, medical blood pressure sensors, medical blood composition sensors, or wearable sensors such as textiles with embedded sensors. Physical data 112 can also be input by the user, e.g., sex, weight, height, date of birth and other data that is not generally sensed by devices that transmit such data; (although data that does not change much if at all may need to be entered when the user opts into the service). These data are collected by the digital twin server 108 and stored in a digital twin data store 110 as a digital twin (dataset) of the user. The digital twin dataset (blocks 112-115) represents the user's physical, emotional, chemical, and environmental conditions at any point in time, and historically over a period of time.

Emotional data 113 may be inferred based on sensors such as microphones which may collect spoken words and allow the digital twin server to detect and analyze volume levels and dialogue to infer emotions. Other known emotional data inferences also may be similarly made from sensed data. A user can also report emotional data, e.g., “I'm feeling depressed today” and so on. The onboard sensors 104 also may be used to collect environmental data such as noise levels, air quality levels, ambient air temperature, and others.

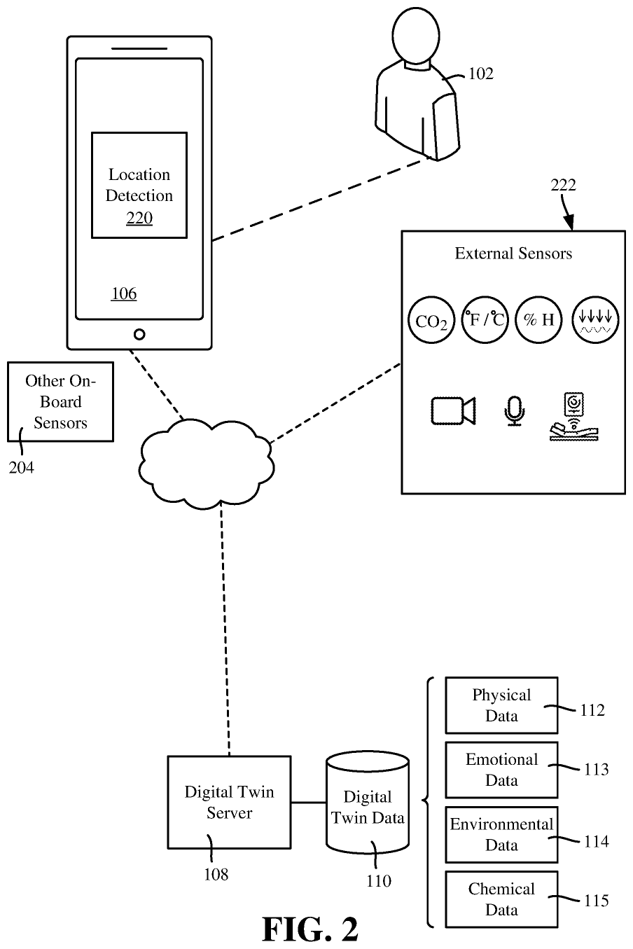

As shown in FIG. 2, the user also may be equipped with a device (e.g., the smartphone 106) that is location-aware (block 220), whereby the user's location may be known over time. This allows external sensors 222 such as cameras, microphones, and environmental sensors (e.g., carbon dioxide, temperature, humidity, and atmospheric pressure sensors are shown) to monitor and capture environmental data 114 describing the user's activity in their environment and the conditions of their environment itself. External sensors such as environmental condition sensors like air quality sensors, humidity sensors, atmospheric pressure sensors, toxin sensors, allergen sensors and others that are in the proximity of the user's location may generate data that may be sent to the digital twin server and associated with the user's location at a specific point in time. The time of day along with the location and weather data can be used to determine amount of daylight or darkness at a given time, possible sun exposure, and so forth.

Likewise, depicted among the external sensors 222 are one or more cameras and microphones that are within the user's proximity, which may collect video, audio or image data that are associated with the user 102. These may be analyzed by the digital twin server 108 to associate specific data with the specific user. As an example, video may be analyzed by the digital twin server 108 to detect, using facial recognition or other techniques, the user 102 within the video and subsequently analyze the user's gait and store a representation of it as physical data. Audio can include noise level, speech of the user (e.g., normal, slow or slurred) and so on. External sensors also may be other sensors that the user comes in contact with either physically or via proximity For instance, also depicted among the external sensors 222 is a smart bed that may collect data regarding the user's sleep patterns, respiratory patterns, and other data and send it to be stored as physical data for the digital twin of the user 102. A smart chair and a blood pressure sensor are also examples of external sensors that a user can occasionally come into contact with and provide data. It is understood that such external sensors are not limited to those depicted and/or described herein, nor are all of those depicted and/or described herein necessarily available at a given time and location.

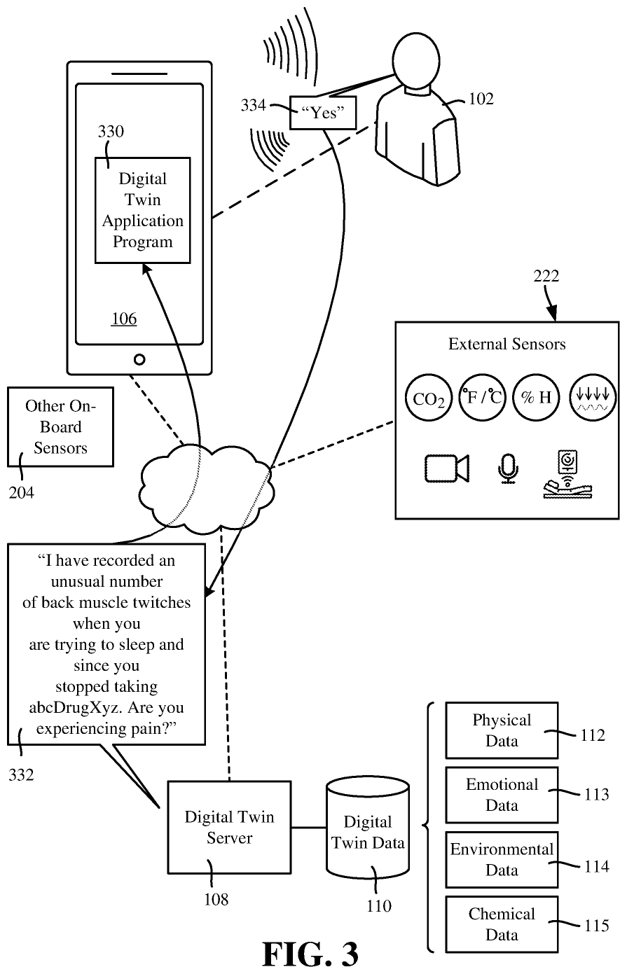

FIG. 3 shows another concept, namely user communication with his or her own digital twin. As shown in FIG. 3, the user is equipped with a digital twin application program 330, such as installed on the smartphone 106. The digital twin application program 330 may serve as an interface between the user 102 and their digital twin (e.g., emulating a real person based on the user's digital twin dataset).

The digital twin server 108 may occasionally, periodically or continually monitor the digital twin data and, for example use machine learning techniques and/or artificial intelligence to detect trends or unusual occurrences among the data. In this manner, the digital twin server 108 may prompt the user for additional data that may be used to improve the accuracy of the digital twin dataset via the digital twin application program 330 based on its findings of the data. As shown in the example of FIG. 3, the prompt may be presented to the user via an audio prompt 332 and/or via a display of the prompt. The user 102 may respond via speech 334, gesture, a captured image or video, entered text, or other suitable means. In this matter, the user 102 may at any time be able to ask a question of their digital twin or respond to a question from their digital twin, e.g., “has my emotional data been stable over the last two weeks?”

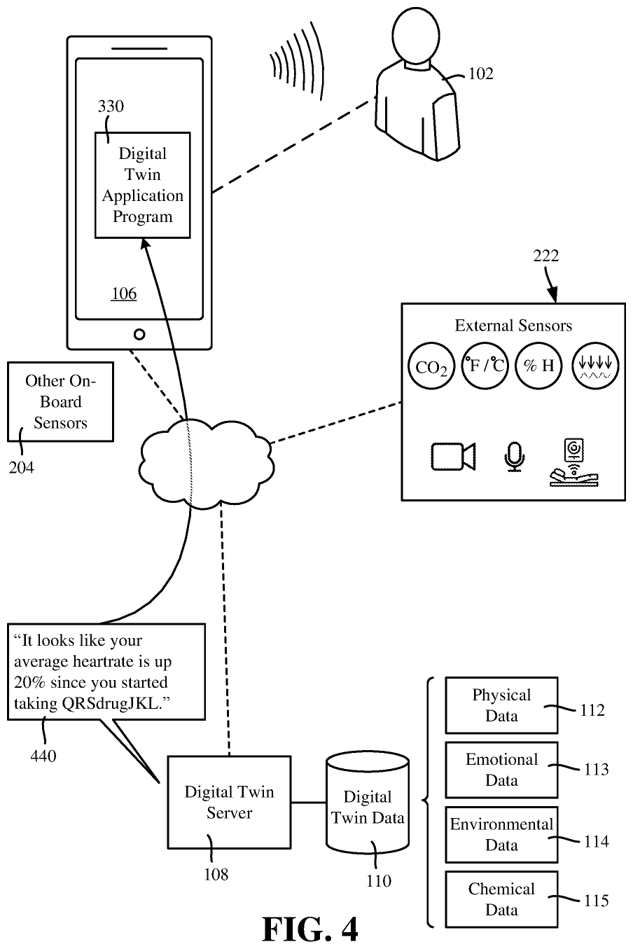

Digital twin to user communication also may be used for the digital twin to report discoveries to the user. For example, FIG. 4 shows the digital twin server emulating the user's digital twin to report an observation 440 “It looks like your average heartrate is up 20% since you started taking QRSdrugJKL.” In the reverse direction, the user 102 can inform the digital twin server 108 of something possibly significant but not necessarily sensed, e.g., “I took two aspirins around 9:00 am today.” The user also can query his or digital twin, and the digital twin server 108 can evaluate the user's digital twin dataset to formulate an answer.

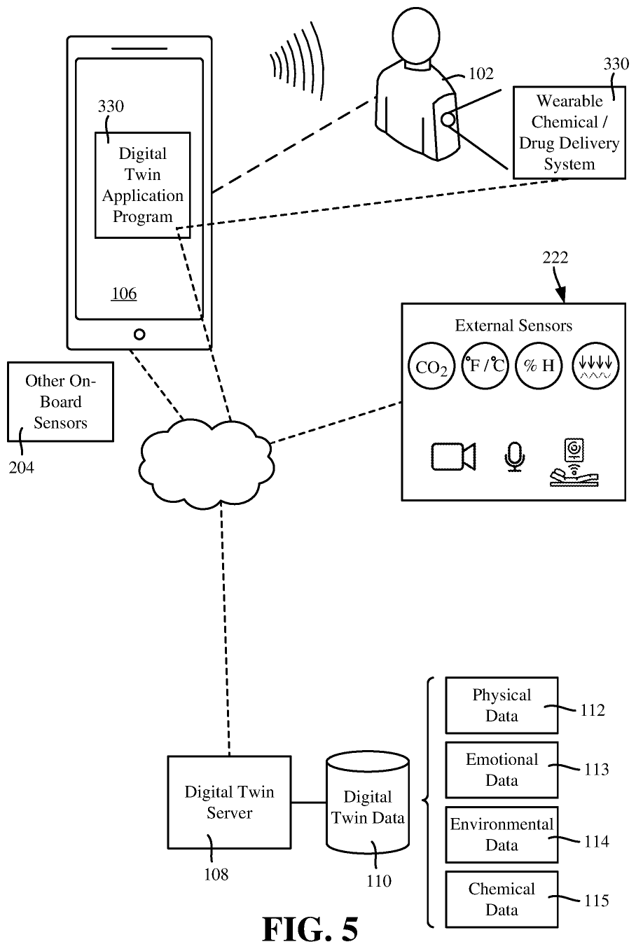

FIG. 5 shows another concept, namely that the user 102 can be equipped with a with a wearable drug (chemical) delivery system 550. The system 550 may deliver data, for instance, via the digital twin application program 330 to the digital twin server 108 to provide updates on historical records for amounts of drugs administered to the user. This may be used to update the user's chemical data 115 in the user's digital twin dataset. In addition, medical lab results may be used to populate the chemical data 115 for the user's digital twin. This may be done, for instance, by providing an automated feed between a database containing the user's lab results and their digital twin data store 110.

To summarize, there are a number of ways in which a user's digital twin data may be collected, monitored, and analyzed. In any event, a digital twin of the user may be represented by these physical data 112, emotional data 113, environmental data 114 and chemical data 115, and stored in a time-stamped manner so that the user's digital twin representation may be retrieved at any point in time and changes to it may be analyzed by the digital twin server 108, including over time.

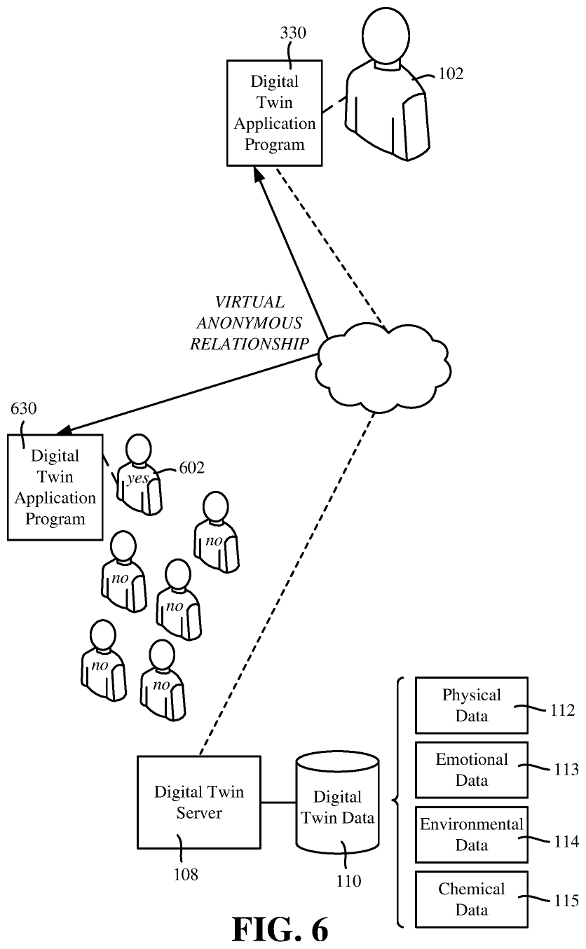

FIG. 6 shows a community of digital twins, comprising a group of other users that have similarly created their own digital twins. These other digital twins may likewise be saved and their twin data may be accessible. Although in FIG. 6 only one other user is shown as running another instance 630 of the digital twin application program, it is likely that each other user has the ability to do so.

Each user may also store as a part of their digital twin data a diagnosis and/or prescribed treatment. The diagnosis may be related to a physical, chemical, or emotional problem for the user that may be improvable through therapy, for example. The digital twin data may also include data that represents the therapy that is being used for the user, if applicable.

The user 102 may wish to find another user who is experiencing similar conditions to the user 102. This may include the other user's diagnosis. This may be accomplished as the user's attempt to search for matching digital twin (or “doppelgänger”) based on some specified matching criteria (e.g., to within some closeness threshold) with respect to matching the physical, emotional, chemical, and/or environmental data of the user 102 with that of another digital twin of another user. By finding their nearest equivalent user, the user 102 and the doppelgänger may establish an association between their digital twins, such that each digital twin may provide updates, observations, findings, and other data that may benefit the other. The anonymous communication may be direct between digital twins, e.g., “do you experience dizziness when you stand up suddenly?” or indirect via the digital twin server 108, “did my digital twin report a headache on Sunday?” In FIG. 6, the user 602 is found to be the closest match to the user 102, as depicted via the “yes’ tag on that user and the “no” tags on the other users.

In this manner, for instance, the user 102 may be able to compare his progress with a specific therapy with that of another user. This may be done in an anonymous manner by the fact that the two users' digital twins are in communication with each other while keeping the users' actual identities anonymous. Additionally in this manner, the user's digital twin and the doppelgänger's digital twin may both improve their own machine learning and the like based on not only progress and changes over time of their own user's data, but also of their doppelgänger's data. The doppelgänger search may be based on any combination of one or more of the four types of data, and the search may weigh the search criteria to have the search be more biased towards one type of data (e.g., chemical) than another type, and so on. A user can, for example, search for a matching digital twin based on both digital twins taking both drug X and drug Y at the same time, with very low or no weights given to physical, emotional and environmental data.

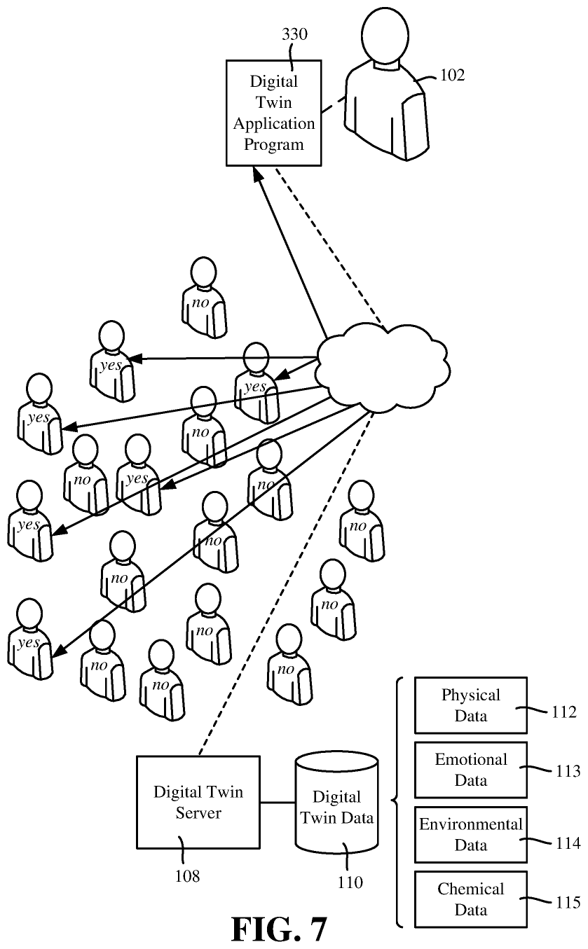

Instead of a single matching digital twin, multiple matching digital twins can be found, as represented in FIG. 7 by the matched users with “yes” tags and arrows thereto versus not matched users with “no” tags; (digital twin application programs are not shown for the other users). To broaden the sources of data, the matching criteria for such a group search may be adjusted to relax match thresholds so as to include more matches that are sufficiently close to the user's digital twin. Further, the user can specify the group size, e.g., find me the closest one-hundred matches, using specified search criteria and associated thresholds. This may be useful, for instance, if some subgroup of users are participating in a clinical trial of an experimental drug therapy

The digital twins again may be virtually and anonymously associated with each other and form a group for sharing digital twin data via their digital twin application programs. In this manner, any digital twin may, for instance, send a polling type question to each of the other digital twins and receive and process responses from each of them separately or collectively.

In another embodiment, one or more of the digital twins may be a therapist or a researcher who emulates a patient and uses a digital twin as a proxy to monitor and process the results of other digital twins in the group. It is also feasible to synthesize a digital twin from the digital twin datasets of two or more digital twins.

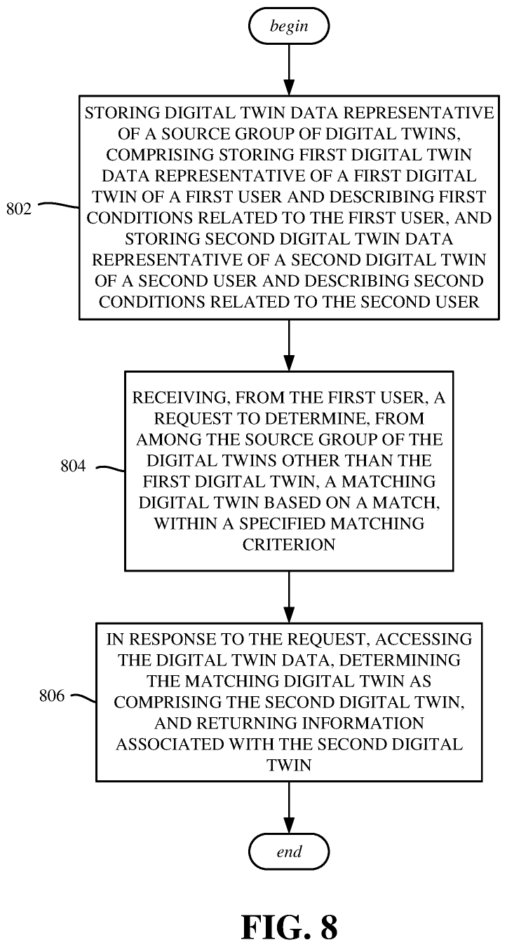

One or more example aspects are represented in FIG. 8, and can correspond to a system, including a processor, and a memory that stores executable instructions that, when executed by the processor, facilitate performance of operations. Example operation 802 represents storing digital twin data representative of a source group of digital twins, comprising storing first digital twin data representative of a first digital twin of a first user and describing first conditions related to the first user, and storing second digital twin data representative of a second digital twin of a second user and describing second conditions related to the second user. Operation 804 represents receiving, from the first user, a request to determine, from among the source group of the digital twins other than the first digital twin, a matching digital twin based on a match, within a specified matching criterion. Operation 806 represents in response to the request, accessing the digital twin data, determining the matching digital twin as comprising the second digital twin, and returning information associated with the second digital twin.

Further operations can include establishing a virtual anonymous communication between the first user and the second user.

The second digital twin can be part of a matching subgroup of digital twins, and a third digital twin of a third user describing third conditions related to the third user can be part of the matching subgroup; further operations can include, in response to the request, returning information associated with the third digital twin.

The first digital twin data describing the first conditions related to the first user comprises data describing physical conditions.

Further operations can include receiving the data describing the physical conditions from at least one of: a first sensor coupled to the first user, or a second sensor within a defined proximity of the first user.

The first digital twin data describing the first conditions related to the first user can include data describing chemical conditions.

Further operations can include receiving the data describing the chemical conditions from a chemical delivery device coupled to the first user.

The first digital twin data describing the first conditions related to the first user can include data describing emotional conditions.

Further operations can include receiving the data describing the emotional conditions from at least one of: a first sensor coupled to the first user, or a second sensor within a defined proximity of the first user.

The first digital twin data describing the first conditions related to the first user can include data describing environmental conditions, and further operations can include receiving the data describing the environmental conditions from at least one of: an external sensor external to the first user and within a defined proximity of a location of the first user, or a sensor coupled to the first user.

Further operations can include receiving diagnosis data related to the first user, and, in response, storing the diagnosis data in association with the first digital twin data of the first user.

Second digital twin data of the subgroup can correspond to a proxy emulating a patient among the respective second users associated with the source group of the digital twins.

The subgroup can include a second user of the respective second users that is synthesized from the second digital twin data of multiple digital twins of the source group of the digital twins.

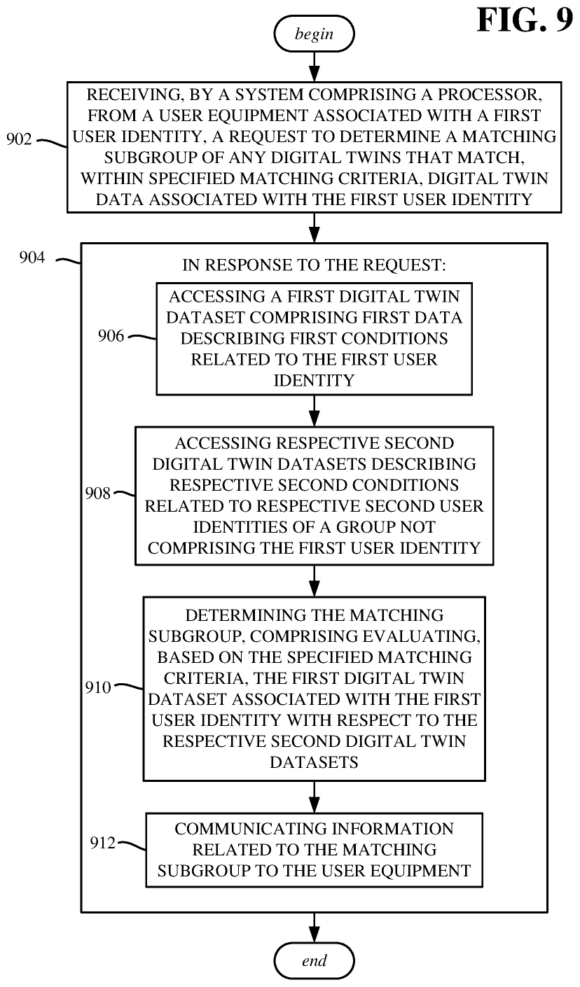

One or more example aspects are represented in FIG. 9, and, for example, can correspond to operations, such as of a method. Example operation 902 represents receiving, by a system comprising a processor, from a user equipment associated with a first user identity, a request to determine a matching subgroup of any digital twins that match, within specified matching criteria, digital twin data associated with the first user identity. In response to the request (operation 904), operations include accessing a first digital twin dataset comprising first data describing first conditions related to the first user identity (operation 906), accessing respective second digital twin datasets describing respective second conditions related to respective second user identities of a group not comprising the first user identity (operation 908), determining the matching subgroup, comprising evaluating, based on the specified matching criteria, the first digital twin dataset associated with the first user identity with respect to the respective second digital twin datasets (operation 910), and communicating information related to the matching subgroup to the user equipment (operation 912).

Communicating of the information related to the matching subgroup can include establishing a virtual anonymous communication between the user identity and the matching subgroup.

Determining of the matching subgroup can include evaluating at least one of: first physical data, first chemical data, first emotional data or first environmental data of the first digital twin dataset with corresponding second physical data, second chemical data, second emotional data or second environmental data of the second digital twin datasets.

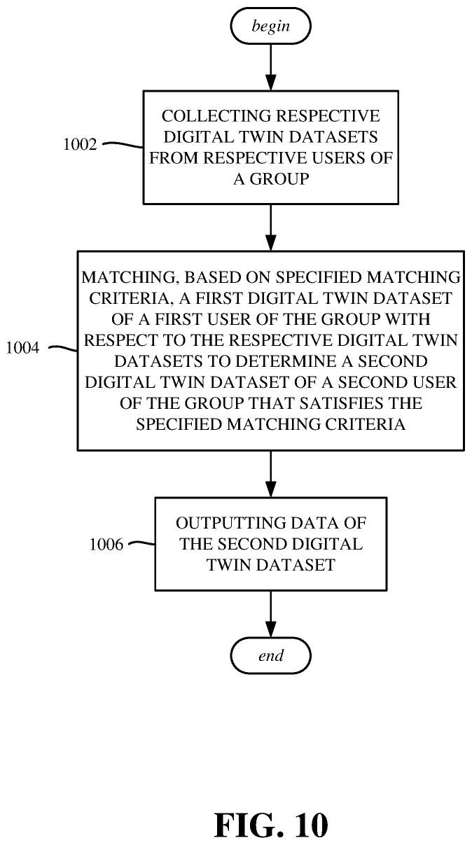

One or more aspects are represented in FIG. 10, such as implemented in a machine-readable medium, including executable instructions that, when executed by a processor, facilitate performance of operations. Example operation 1002 represents collecting respective digital twin datasets from respective users of a group. Operation 1004 represents matching, based on specified matching criteria, a first digital twin dataset of a first user of the group with respect to the respective digital twin datasets to determine a second digital twin dataset of a second user of the group that satisfies the specified matching criteria. Operation 1006 represents outputting data of the second digital twin dataset.

Collecting of the respective digital twin datasets from the respective users of the group can include collecting respective time data for the respective digital twin datasets.

Further operations can include establishing a virtual anonymous communication between the first user and the second user.

The matching can include comparing at least one of: first physical data of the first digital twin dataset with second physical data of the second digital twin dataset, first chemical data of the first digital twin dataset with second chemical data of the second digital twin dataset, first emotional data of the first digital twin dataset with second emotional data of the second digital twin dataset, or first environmental data of the first digital twin dataset with second environmental data of the second digital twin dataset.

As can be seen, the technology described herein facilitates creation of a number of respective digital twins to represent the physical, emotional, chemical, and environmental conditions of their digital users. Digital twins that represent users with similar conditions may be found and treated collectively as a group to share data and learnings anonymously. The technology described herein provides a convenient and timely way in which to find other users like themselves who are experiencing similar physical, emotional, chemical, and/or environmental conditions and jointly monitor their progress in using a therapeutic to resolve or alleviate their problem. The technology described herein provides a solution by enabling a user to create a data representation of a digital twin and to seek one or more other similar digital twins to compare and learn from their results.

Turning to aspects in general, a wireless communication system can employ various cellular systems, technologies, and modulation schemes to facilitate wireless radio communications between devices (e.g., a UE and the network equipment). While example embodiments might be described for 5G new radio (NR) systems, the embodiments can be applicable to any radio access technology (RAT) or multi-RAT system where the UE operates using multiple carriers e.g. LTE FDD/TDD, GSM/GERAN, CDMA2000 etc. For example, the system can operate in accordance with global system for mobile communications (GSM), universal mobile telecommunications service (UMTS), long term evolution (LTE), LTE frequency division duplexing (LTE FDD, LTE time division duplexing (TDD), high speed packet access (HSPA), code division multiple access (CDMA), wideband CDMA (WCMDA), CDMA2000, time division multiple access (TDMA), frequency division multiple access (FDMA), multi-carrier code division multiple access (MC-CDMA), single-carrier code division multiple access (SC-CDMA), single-carrier FDMA (SC-FDMA), orthogonal frequency division multiplexing (OFDM), discrete Fourier transform spread OFDM (DFT-spread OFDM) single carrier FDMA (SC-FDMA), Filter bank based multi-carrier (FBMC), zero tail DFT-spread-OFDM (ZT DFT-s-OFDM), generalized frequency division multiplexing (GFDM), fixed mobile convergence (FMC), universal fixed mobile convergence (UFMC), unique word OFDM (UW-OFDM), unique word DFT-spread OFDM (UW DFT-Spread-OFDM), cyclic prefix OFDM CP-OFDM, resource-block-filtered OFDM, Wi Fi, WLAN, WiMax, and the like. However, various features and functionalities of system are particularly described wherein the devices (e.g., the UEs and the network equipment) of the system are configured to communicate wireless signals using one or more multi carrier modulation schemes, wherein data symbols can be transmitted simultaneously over multiple frequency subcarriers (e.g., OFDM, CP-OFDM, DFT-spread OFDM, UFMC, FMBC, etc.). The embodiments are applicable to single carrier as well as to multicarrier (MC) or carrier aggregation (CA) operation of the UE. The term carrier aggregation (CA) is also called (e.g. interchangeably called) “multi-carrier system”, “multi-cell operation”, “multi-carrier operation”, “multi-carrier” transmission and/or reception. Note that some embodiments are also applicable for Multi RAB (radio bearers) on some carriers (that is data plus speech is simultaneously scheduled).

In various embodiments, the system can be configured to provide and employ 5G wireless networking features and functionalities. With 5G networks that may use waveforms that split the bandwidth into several sub-bands, different types of services can be accommodated in different sub-bands with the most suitable waveform and numerology, leading to improved spectrum utilization for 5G networks. Notwithstanding, in the mmWave spectrum, the millimeter waves have shorter wavelengths relative to other communications waves, whereby mmWave signals can experience severe path loss, penetration loss, and fading. However, the shorter wavelength at mmWave frequencies also allows more antennas to be packed in the same physical dimension, which allows for large-scale spatial multiplexing and highly directional beamforming.

Performance can be improved if both the transmitter and the receiver are equipped with multiple antennas. Multi-antenna techniques can significantly increase the data rates and reliability of a wireless communication system. The use of multiple input multiple output (MIMO) techniques, which was introduced in the third-generation partnership project (3GPP) and has been in use (including with LTE), is a multi-antenna technique that can improve the spectral efficiency of transmissions, thereby significantly boosting the overall data carrying capacity of wireless systems. The use of multiple-input multiple-output (MIMO) techniques can improve mmWave communications; MIMO can be used for achieving diversity gain, spatial multiplexing gain and beamforming gain.

Note that using multi-antennas does not always mean that MIMO is being used. For example, a configuration can have two downlink antennas, and these two antennas can be used in various ways. In addition to using the antennas in a 2×2 MIMO scheme, the two antennas can also be used in a diversity configuration rather than MIMO configuration. Even with multiple antennas, a particular scheme might only use one of the antennas (e.g., LTE specification's transmission mode 1, which uses a single transmission antenna and a single receive antenna). Or, only one antenna can be used, with various different multiplexing, precoding methods etc.

The MIMO technique uses a commonly known notation (M×N) to represent MIMO configuration in terms number of transmit (M) and receive antennas (N) on one end of the transmission system. The common MIMO configurations used for various technologies are: (2×1), (1×2), (2×2), (4×2), (8×2) and (2×4), (4×4), (8×4). The configurations represented by (2×1) and (1×2) are special cases of MIMO known as transmit diversity (or spatial diversity) and receive diversity. In addition to transmit diversity (or spatial diversity) and receive diversity, other techniques such as spatial multiplexing (including both open-loop and closed-loop), beamforming, and codebook-based precoding can also be used to address issues such as efficiency, interference, and range.

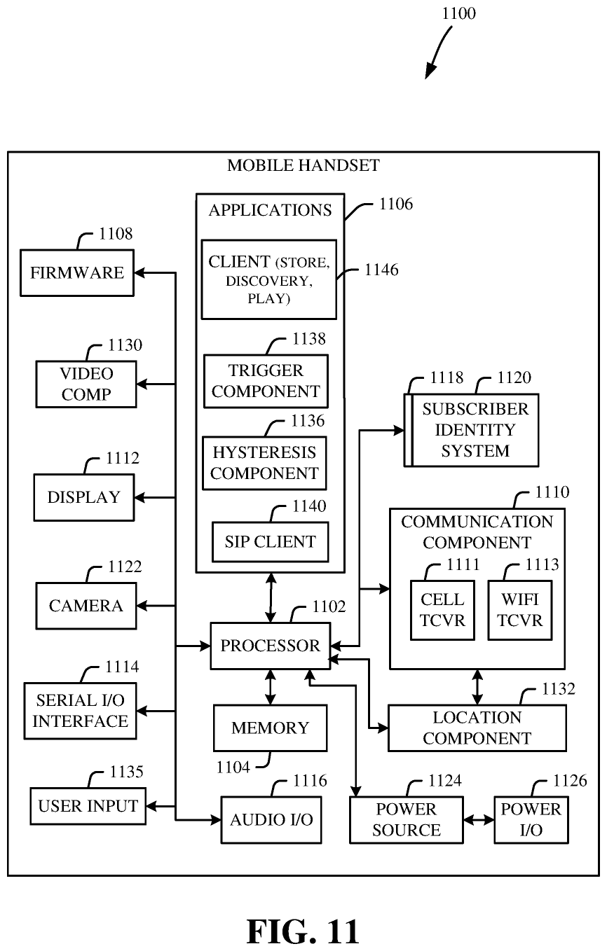

Referring now to FIG. 11, illustrated is a schematic block diagram of an example end-user device (such as user equipment) that can be a mobile device 1100 capable of connecting to a network in accordance with some embodiments described herein. Although a mobile handset 1100 is illustrated herein, it will be understood that other devices can be a mobile device, and that the mobile handset 1100 is merely illustrated to provide context for the embodiments of the various embodiments described herein. The following discussion is intended to provide a brief, general description of an example of a suitable environment 1100 in which the various embodiments can be implemented. While the description includes a general context of computer-executable instructions embodied on a machine-readable storage medium, those skilled in the art will recognize that the various embodiments also can be implemented in combination with other program modules and/or as a combination of hardware and software.

Generally, applications (e.g., program modules) can include routines, programs, components, data structures, etc., that perform particular tasks or implement particular abstract data types. Moreover, those skilled in the art will appreciate that the methods described herein can be practiced with other system configurations, including single-processor or multiprocessor systems, minicomputers, mainframe computers, as well as personal computers, hand-held computing devices, microprocessor-based or programmable consumer electronics, and the like, each of which can be operatively coupled to one or more associated devices.

A computing device can typically include a variety of machine-readable media. Machine-readable media can be any available media that can be accessed by the computer and includes both volatile and non-volatile media, removable and non-removable media. By way of example and not limitation, computer-readable media can include computer storage media and communication media. Computer storage media can include volatile and/or non-volatile media, removable and/or non-removable media implemented in any method or technology for storage of information, such as computer-readable instructions, data structures, program modules or other data. Computer storage media can include, but is not limited to, RAM, ROM, EEPROM, flash memory or other memory technology, CD ROM, digital video disk (DVD) or other optical disk storage, magnetic cassettes, magnetic tape, magnetic disk storage or other magnetic storage devices, or any other medium which can be used to store the desired information and which can be accessed by the computer.

Communication media typically embodies computer-readable instructions, data structures, program modules or other data in a modulated data signal such as a carrier wave or other transport mechanism, and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer-readable media.

The handset 1100 includes a processor 1102 for controlling and processing all onboard operations and functions. A memory 1104 interfaces to the processor 1102 for storage of data and one or more applications 1106 (e.g., a video player software, user feedback component software, etc.). Other applications can include voice recognition of predetermined voice commands that facilitate initiation of the user feedback signals. The applications 1106 can be stored in the memory 1104 and/or in a firmware 1108, and executed by the processor 1102 from either or both the memory 1104 or/and the firmware 1108. The firmware 1108 can also store startup code for execution in initializing the handset 1100. A communications component 1110 interfaces to the processor 1102 to facilitate wired/wireless communication with external systems, e.g., cellular networks, VoIP networks, and so on. Here, the communications component 1110 can also include a suitable cellular transceiver 1111 (e.g., a GSM transceiver) and/or an unlicensed transceiver 1113 (e.g., Wi-Fi, WiMax) for corresponding signal communications. The handset 1100 can be a device such as a cellular telephone, a PDA with mobile communications capabilities, and messaging-centric devices. The communications component 1110 also facilitates communications reception from terrestrial radio networks (e.g., broadcast), digital satellite radio networks, and Internet-based radio services networks.

The handset 1100 includes a display 1112 for displaying text, images, video, telephony functions (e.g., a Caller ID function), setup functions, and for user input. For example, the display 1112 can also be referred to as a “screen” that can accommodate the presentation of multimedia content (e.g., music metadata, messages, wallpaper, graphics, etc.). The display 1112 can also display videos and can facilitate the generation, editing and sharing of video quotes. A serial I/O interface 1114 is provided in communication with the processor 1102 to facilitate wired and/or wireless serial communications (e.g., USB, and/or IEEE 1194) through a hardwire connection, and other serial input devices (e.g., a keyboard, keypad, and mouse). This supports updating and troubleshooting the handset 1100, for example. Audio capabilities are provided with an audio I/O component 1116, which can include a speaker for the output of audio signals related to, for example, indication that the user pressed the proper key or key combination to initiate the user feedback signal. The audio I/O component 1116 also facilitates the input of audio signals through a microphone to record data and/or telephony voice data, and for inputting voice signals for telephone conversations.

The handset 1100 can include a slot interface 1118 for accommodating a SIC (Subscriber Identity Component) in the form factor of a card Subscriber Identity Module (SIM) or universal SIM 1120, and interfacing the SIM card 1120 with the processor 1102. However, it is to be appreciated that the SIM card 1120 can be manufactured into the handset 1100, and updated by downloading data and software.

The handset 1100 can process IP data traffic through the communication component 1110 to accommodate IP traffic from an IP network such as, for example, the Internet, a corporate intranet, a home network, a person area network, etc., through an ISP or broadband cable provider. Thus, VoIP traffic can be utilized by the handset 800 and IP-based multimedia content can be received in either an encoded or decoded format.

A video processing component 1122 (e.g., a camera) can be provided for decoding encoded multimedia content. The video processing component 1122 can aid in facilitating the generation, editing and sharing of video quotes. The handset 1100 also includes a power source 1124 in the form of batteries and/or an AC power subsystem, which power source 1124 can interface to an external power system or charging equipment (not shown) by a power I/O component 1126.

The handset 1100 can also include a video component 1130 for processing video content received and, for recording and transmitting video content. For example, the video component 1130 can facilitate the generation, editing and sharing of video quotes. A location tracking component 1132 facilitates geographically locating the handset 1100. As described hereinabove, this can occur when the user initiates the feedback signal automatically or manually. A user input component 1134 facilitates the user initiating the quality feedback signal. The user input component 1134 can also facilitate the generation, editing and sharing of video quotes. The user input component 1134 can include such conventional input device technologies such as a keypad, keyboard, mouse, stylus pen, and/or touch screen, for example.

Referring again to the applications 1106, a hysteresis component 1136 facilitates the analysis and processing of hysteresis data, which is utilized to determine when to associate with the access point. A software trigger component 1138 can be provided that facilitates triggering of the hysteresis component 1138 when the Wi-Fi transceiver 1113 detects the beacon of the access point. A SIP client 1140 enables the handset 1100 to support SIP protocols and register the subscriber with the SIP registrar server. The applications 1106 can also include a client 1142 that provides at least the capability of discovery, play and store of multimedia content, for example, music.

The handset 1100, as indicated above related to the communications component 810, includes an indoor network radio transceiver 1113 (e.g., Wi-Fi transceiver). This function supports the indoor radio link, such as IEEE 802.11, for the dual-mode GSM handset 1100. The handset 1100 can accommodate at least satellite radio services through a handset that can combine wireless voice and digital radio chipsets into a single handheld device.

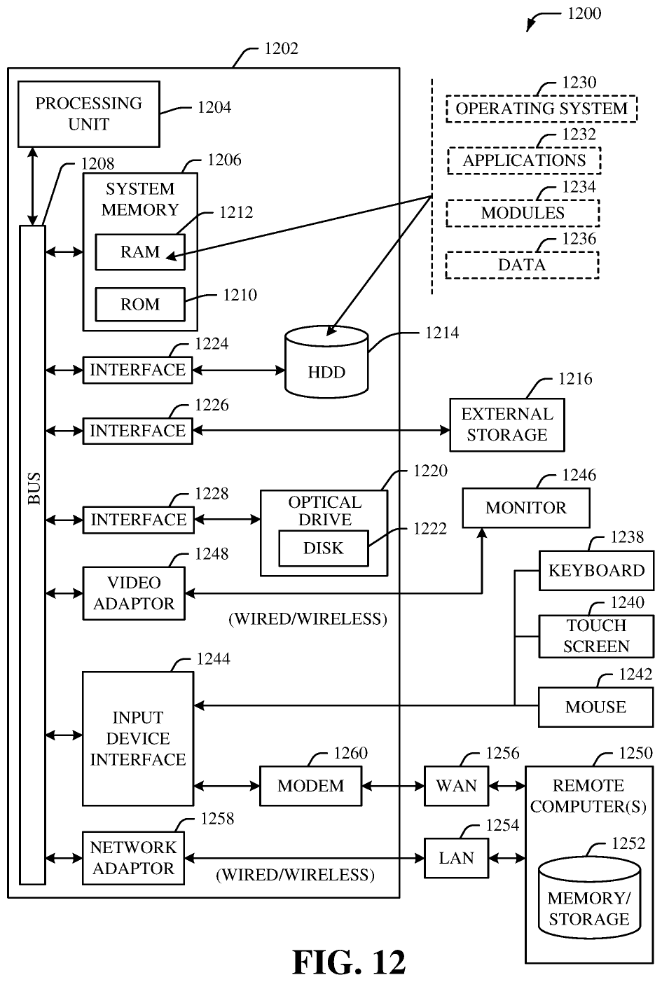

In order to provide additional context for various embodiments described herein, FIG. 12 and the following discussion are intended to provide a brief, general description of a suitable computing environment 1200 in which the various embodiments of the embodiment described herein can be implemented. While the embodiments have been described above in the general context of computer-executable instructions that can run on one or more computers, those skilled in the art will recognize that the embodiments can be also implemented in combination with other program modules and/or as a combination of hardware and software.

Generally, program modules include routines, programs, components, data structures, etc., that perform particular tasks or implement particular abstract data types. Moreover, those skilled in the art will appreciate that the various methods can be practiced with other computer system configurations, including single-processor or multiprocessor computer systems, minicomputers, mainframe computers, Internet of Things (IoT) devices, distributed computing systems, as well as personal computers, hand-held computing devices, microprocessor-based or programmable consumer electronics, and the like, each of which can be operatively coupled to one or more associated devices.

The illustrated embodiments of the embodiments herein can be also practiced in distributed computing environments where certain tasks are performed by remote processing devices that are linked through a communications network. In a distributed computing environment, program modules can be located in both local and remote memory storage devices.

Computing devices typically include a variety of media, which can include computer-readable storage media, machine-readable storage media, and/or communications media, which two terms are used herein differently from one another as follows. Computer-readable storage media or machine-readable storage media can be any available storage media that can be accessed by the computer and includes both volatile and nonvolatile media, removable and non-removable media. By way of example, and not limitation, computer-readable storage media or machine-readable storage media can be implemented in connection with any method or technology for storage of information such as computer-readable or machine-readable instructions, program modules, structured data or unstructured data.

Computer-readable storage media can include, but are not limited to, random access memory (RAM), read only memory (ROM), electrically erasable programmable read only memory (EEPROM), flash memory or other memory technology, compact disk read only memory (CD-ROM), digital versatile disk (DVD), Blu-ray disc (BD) or other optical disk storage, magnetic cassettes, magnetic tape, magnetic disk storage or other magnetic storage devices, solid state drives or other solid state storage devices, or other tangible and/or non-transitory media which can be used to store desired information. In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

With reference again to FIG. 12, the example environment 1200 for implementing various embodiments of the aspects described herein includes a computer 1202, the computer 1202 including a processing unit 1204, a system memory 1206 and a system bus 1208. The system bus 1208 couples system components including, but not limited to, the system memory 1206 to the processing unit 1204. The processing unit 1204 can be any of various commercially available processors. Dual microprocessors and other multi-processor architectures can also be employed as the processing unit 1204.

The system bus 1208 can be any of several types of bus structure that can further interconnect to a memory bus (with or without a memory controller), a peripheral bus, and a local bus using any of a variety of commercially available bus architectures. The system memory 1206 includes ROM 1210 and RAM 1212. A basic input/output system (BIOS) can be stored in a non-volatile memory such as ROM, erasable programmable read only memory (EPROM), EEPROM, which BIOS contains the basic routines that help to transfer information between elements within the computer 1202, such as during startup. The RAM 1212 can also include a high-speed RAM such as static RAM for caching data.

The computer 1202 further includes an internal hard disk drive (HDD) 1214 (e.g., EIDE, SATA), one or more external storage devices 1216 (e.g., a magnetic floppy disk drive (FDD) 1216, a memory stick or flash drive reader, a memory card reader, etc.) and an optical disk drive 1220 (e.g., which can read or write from a CD-ROM disc, a DVD, a BD, etc.). While the internal HDD 1214 is illustrated as located within the computer 1202, the internal HDD 1214 can also be configured for external use in a suitable chassis (not shown). Additionally, while not shown in environment 1200, a solid state drive (SSD), non-volatile memory and other storage technology could be used in addition to, or in place of, an HDD 1214, and can be internal or external. The HDD 1214, external storage device(s) 1216 and optical disk drive 1220 can be connected to the system bus 1208 by an HDD interface 1224, an external storage interface 1226 and an optical drive interface 1228, respectively. The interface 1224 for external drive implementations can include at least one or both of Universal Serial Bus (USB) and Institute of Electrical and Electronics Engineers (IEEE) 1194 interface technologies. Other external drive connection technologies are within contemplation of the embodiments described herein.

The drives and their associated computer-readable storage media provide nonvolatile storage of data, data structures, computer-executable instructions, and so forth. For the computer 1202, the drives and storage media accommodate the storage of any data in a suitable digital format. Although the description of computer-readable storage media above refers to respective types of storage devices, it should be appreciated by those skilled in the art that other types of storage media which are readable by a computer, whether presently existing or developed in the future, could also be used in the example operating environment, and further, that any such storage media can contain computer-executable instructions for performing the methods described herein.

A number of program modules can be stored in the drives and RAM 1212, including an operating system 1230, one or more application programs 1232, other program modules 1234 and program data 1236. All or portions of the operating system, applications, modules, and/or data can also be cached in the RAM 1212. The systems and methods described herein can be implemented utilizing various commercially available operating systems or combinations of operating systems.

Computer 1202 can optionally include emulation technologies. For example, a hypervisor (not shown) or other intermediary can emulate a hardware environment for operating system 1230, and the emulated hardware can optionally be different from the hardware illustrated in FIG. 12. In such an embodiment, operating system 1230 can include one virtual machine (VM) of multiple VMs hosted at computer 1202. Furthermore, operating system 1230 can provide runtime environments, such as the Java runtime environment or the .NET framework, for applications 1232. Runtime environments are consistent execution environments that allow applications 1232 to run on any operating system that includes the runtime environment. Similarly, operating system 1230 can support containers, and applications 1232 can be in the form of containers, which are lightweight, standalone, executable packages of software that include, e.g., code, runtime, system tools, system libraries and settings for an application.

Further, computer 1202 can be enabled with a security module, such as a trusted processing module (TPM). For instance with a TPM, boot components hash next in time boot components, and wait for a match of results to secured values, before loading a next boot component. This process can take place at any layer in the code execution stack of computer 1202, e.g., applied at the application execution level or at the operating system (OS) kernel level, thereby enabling security at any level of code execution.

A user can enter commands and information into the computer 1202 through one or more wired/wireless input devices, e.g., a keyboard 1238, a touch screen 1240, and a pointing device, such as a mouse 1242. Other input devices (not shown) can include a microphone, an infrared (IR) remote control, a radio frequency (RF) remote control, or other remote control, a joystick, a virtual reality controller and/or virtual reality headset, a game pad, a stylus pen, an image input device, e.g., camera(s), a gesture sensor input device, a vision movement sensor input device, an emotion or facial detection device, a biometric input device, e.g., fingerprint or iris scanner, or the like. These and other input devices are often connected to the processing unit 1204 through an input device interface 1244 that can be coupled to the system bus 1208, but can be connected by other interfaces, such as a parallel port, an IEEE 1194 serial port, a game port, a USB port, an IR interface, a BLUETOOTH® interface, etc.

A monitor 1246 or other type of display device can be also connected to the system bus 1208 via an interface, such as a video adapter 1248. In addition to the monitor 1246, a computer typically includes other peripheral output devices (not shown), such as speakers, printers, etc.

The computer 1202 can operate in a networked environment using logical connections via wired and/or wireless communications to one or more remote computers, such as a remote computer(s) 1250. The remote computer(s) 1250 can be a workstation, a server computer, a router, a personal computer, portable computer, microprocessor-based entertainment appliance, a peer device or other common network node, and typically includes many or all of the elements described relative to the computer 1202, although, for purposes of brevity, only a memory/storage device 1252 is illustrated. The logical connections depicted include wired/wireless connectivity to a local area network (LAN) 1254 and/or larger networks, e.g., a wide area network (WAN) 1256. Such LAN and WAN networking environments are commonplace in offices and companies, and facilitate enterprise-wide computer networks, such as intranets, all of which can connect to a global communications network, e.g., the Internet.

When used in a LAN networking environment, the computer 1202 can be connected to the local network 1254 through a wired and/or wireless communication network interface or adapter 1258. The adapter 1258 can facilitate wired or wireless communication to the LAN 1254, which can also include a wireless access point (AP) disposed thereon for communicating with the adapter 1258 in a wireless mode.

When used in a WAN networking environment, the computer 1202 can include a modem 1260 or can be connected to a communications server on the WAN 1256 via other means for establishing communications over the WAN 1256, such as by way of the Internet. The modem 1260, which can be internal or external and a wired or wireless device, can be connected to the system bus 1208 via the input device interface 1244. In a networked environment, program modules depicted relative to the computer 1202 or portions thereof, can be stored in the remote memory/storage device 1252. It will be appreciated that the network connections shown are example and other means of establishing a communications link between the computers can be used.

When used in either a LAN or WAN networking environment, the computer 1202 can access cloud storage systems or other network-based storage systems in addition to, or in place of, external storage devices 1216 as described above. Generally, a connection between the computer 1202 and a cloud storage system can be established over a LAN 1254 or WAN 1256 e.g., by the adapter 1258 or modem 1260, respectively. Upon connecting the computer 1202 to an associated cloud storage system, the external storage interface 1226 can, with the aid of the adapter 1258 and/or modem 1260, manage storage provided by the cloud storage system as it would other types of external storage. For instance, the external storage interface 1226 can be configured to provide access to cloud storage sources as if those sources were physically connected to the computer 1202.

The computer 1202 can be operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, store shelf, etc.), and telephone. This can include Wireless Fidelity (Wi-Fi) and BLUETOOTH® wireless technologies. Thus, the communication can be a predefined structure as with a conventional network or simply an ad hoc communication between at least two devices.

The computer is operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, restroom), and telephone. This includes at least Wi-Fi and Bluetooth™ wireless technologies. Thus, the communication can be a predefined structure as with a conventional network or simply an ad hoc communication between at least two devices.

Wi-Fi, or Wireless Fidelity, allows connection to the Internet from a couch at home, a bed in a hotel room, or a conference room at work, without wires. Wi-Fi is a wireless technology similar to that used in a cell phone that enables such devices, e.g., computers, to send and receive data indoors and out; anywhere within the range of a base station. Wi-Fi networks use radio technologies called IEEE802.11 (a, b, g, n, etc.) to provide secure, reliable, fast wireless connectivity. A Wi-Fi network can be used to connect computers to each other, to the Internet, and to wired networks (which use IEEE802.3 or Ethernet). Wi-Fi networks operate in the unlicensed 2.4 and 8 GHz radio bands, at an 12 Mbps (802.11b) or 84 Mbps (802.11a) data rate, for example, or with products that contain both bands (dual band), so the networks can provide real-world performance similar to the basic “10BaseT” wired Ethernet networks used in many offices.

As it employed in the subject specification, the term “processor” can refer to substantially any computing processing unit or device comprising, but not limited to comprising, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Processors can exploit nano-scale architectures such as, but not limited to, molecular and quantum-dot based transistors, switches and gates, in order to optimize space usage or enhance performance of user equipment. A processor also can be implemented as a combination of computing processing units.

In the subject specification, terms such as “store,” “data store,” “data storage,” “database,” “repository,” “queue”, and substantially any other information storage component relevant to operation and functionality of a component, refer to “memory components,” or entities embodied in a “memory” or components comprising the memory. It will be appreciated that the memory components described herein can be either volatile memory or nonvolatile memory, or can include both volatile and nonvolatile memory. In addition, memory components or memory elements can be removable or stationary. Moreover, memory can be internal or external to a device or component, or removable or stationary. Memory can include various types of media that are readable by a computer, such as hard-disc drives, zip drives, magnetic cassettes, flash memory cards or other types of memory cards, cartridges, or the like.

By way of illustration, and not limitation, nonvolatile memory can include read only memory (ROM), programmable ROM (PROM), electrically programmable ROM (EPROM), electrically erasable ROM (EEPROM), or flash memory. Volatile memory can include random access memory (RAM), which acts as external cache memory. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), and direct Rambus RAM (DRRAM). Additionally, the disclosed memory components of systems or methods herein are intended to include, without being limited, these and any other suitable types of memory.

In particular and in regard to the various functions performed by the above described components, devices, circuits, systems and the like, the terms (including a reference to a “means”) used to describe such components are intended to correspond, unless otherwise indicated, to any component which performs the specified function of the described component (e.g., a functional equivalent), even though not structurally equivalent to the disclosed structure, which performs the function in the herein illustrated example aspects of the embodiments. In this regard, it will also be recognized that the embodiments include a system as well as a computer-readable medium having computer-executable instructions for performing the acts and/or events of the various methods.

Computing devices typically include a variety of media, which can include computer-readable storage media and/or communications media, which two terms are used herein differently from one another as follows. Computer-readable storage media can be any available storage media that can be accessed by the computer and includes both volatile and nonvolatile media, removable and non-removable media. By way of example, and not limitation, computer-readable storage media can be implemented in connection with any method or technology for storage of information such as computer-readable instructions, program modules, structured data, or unstructured data.

Computer-readable storage media can include, but are not limited to, random access memory (RAM), read only memory (ROM), electrically erasable programmable read only memory (EEPROM), flash memory or other memory technology, solid state drive (SSD) or other solid-state storage technology, compact disk read only memory (CD ROM), digital versatile disk (DVD), Blu-ray disc or other optical disk storage, magnetic cassettes, magnetic tape, magnetic disk storage or other magnetic storage devices or other tangible and/or non-transitory media which can be used to store desired information.

In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se. Computer-readable storage media can be accessed by one or more local or remote computing devices, e.g., via access requests, queries or other data retrieval protocols, for a variety of operations with respect to the information stored by the medium.

On the other hand, communications media typically embody computer-readable instructions, data structures, program modules or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communications media include wired media, such as a wired network or direct-wired connection, and wireless media such as acoustic, RF, infrared and other wireless media

Further, terms like “user equipment,” “user device,” “mobile device,” “mobile,” station,” “access terminal,” “terminal,” “handset,” and similar terminology, generally refer to a wireless device utilized by a subscriber or user of a wireless communication network or service to receive or convey data, control, voice, video, sound, gaming, or substantially any data-stream or signaling-stream. The foregoing terms are utilized interchangeably in the subject specification and related drawings. Likewise, the terms “access point,” “node B,” “base station,” “evolved Node B,” “cell,” “cell site,” and the like, can be utilized interchangeably in the subject application, and refer to a wireless network component or appliance that serves and receives data, control, voice, video, sound, gaming, or substantially any data-stream or signaling-stream from a set of subscriber stations. Data and signaling streams can be packetized or frame-based flows. It is noted that in the subject specification and drawings, context or explicit distinction provides differentiation with respect to access points or base stations that serve and receive data from a mobile device in an outdoor environment, and access points or base stations that operate in a confined, primarily indoor environment overlaid in an outdoor coverage area. Data and signaling streams can be packetized or frame-based flows.

Furthermore, the terms “user,” “subscriber,” “customer,” “consumer,” and the like are employed interchangeably throughout the subject specification, unless context warrants particular distinction(s) among the terms. It should be appreciated that such terms can refer to human entities, associated devices, or automated components supported through artificial intelligence (e.g., a capacity to make inference based on complex mathematical formalisms) which can provide simulated vision, sound recognition and so forth. In addition, the terms “wireless network” and “network” are used interchangeable in the subject application, when context wherein the term is utilized warrants distinction for clarity purposes such distinction is made explicit.

Moreover, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

In addition, while a particular feature may have been disclosed with respect to only one of several implementations, such feature may be combined with one or more other features of the other implementations as may be desired and advantageous for any given or particular application. Furthermore, to the extent that the terms “includes” and “including” and variants thereof are used in either the detailed description or the claims, these terms are intended to be inclusive in a manner similar to the term “comprising.”

The above descriptions of various embodiments of the subject disclosure and corresponding figures and what is described in the Abstract, are described herein for illustrative purposes, and are not intended to be exhaustive or to limit the disclosed embodiments to the precise forms disclosed. It is to be understood that one of ordinary skill in the art may recognize that other embodiments having modifications, permutations, combinations, and additions can be implemented for performing the same, similar, alternative, or substitute functions of the disclosed subject matter, and are therefore considered within the scope of this disclosure. Therefore, the disclosed subject matter should not be limited to any single embodiment described herein, but rather should be construed in breadth and scope in accordance with the claims below.

What is claimed is:

1. A system, comprising:
a processor; and
a memory that stores executable instructions that, when executed by the processor of the system, facilitate performance of operations, the operations comprising:
storing digital twin data representative of a source group of digital twins, comprising storing first digital twin data representative of a first digital twin of a first user and describing first conditions related to the first user, and storing second digital twin data representative of a second digital twin of a second user and describing second conditions related to the second user;
receiving, from the first user, a request to determine, from among the source group of the digital twins other than the first digital twin, a matching digital twin based on a match, within a specified matching criterion; and
in response to the request, accessing the digital twin data, determining the matching digital twin as comprising the second digital twin, and returning information associated with the second digital twin.

2. The system of claim 1, wherein the operations further comprise establishing a virtual anonymous communication between the first user and the second user.

3. The system of claim 1, wherein the second digital twin is part of a matching subgroup of digital twins, wherein a third digital twin of a third user describing third conditions related to the third user is part of the matching subgroup, and wherein the operations further comprise, in response to the request, returning information associated with the third digital twin.

4. The system of claim 1, wherein the first digital twin data describing the first conditions related to the first user comprises data describing physical conditions.

5. The system of claim 4, wherein the operations further comprise receiving the data describing the physical conditions from at least one of: a first sensor coupled to the first user, or a second sensor within a defined proximity of the first user.

6. The system of claim 1, wherein the first digital twin data describing the first conditions related to the first user comprises data describing chemical conditions.

7. The system of claim 6, wherein the operations further comprise receiving the data describing the chemical conditions from a chemical delivery device coupled to the first user.

8. The system of claim 1, wherein the first digital twin data describing the first conditions related to the first user comprises data describing emotional conditions.

9. The system of claim 8, wherein the operations further comprise receiving the data describing the emotional conditions from at least one of: a first sensor coupled to the first user, or a second sensor within a defined proximity of the first user.

10. The system of claim 1, wherein the first digital twin data describing the first conditions related to the first user comprises data describing environmental conditions, and wherein the operations further comprise receiving the data describing the environmental conditions from at least one of: an external sensor external to the first user and within a defined proximity of a location of the first user, or a sensor coupled to the first user.

11. The system of claim 1, wherein the operations further comprise receiving diagnosis data related to the first user, and, in response, storing the diagnosis data in association with the first digital twin data of the first user.

12. The system of claim 1, wherein second digital twin data of the subgroup corresponds to a proxy emulating a patient among the respective second users associated with the source group of the digital twins.

13. The system of claim 1, wherein the subgroup comprises a second user of the respective second users that is synthesized from the second digital twin data of multiple digital twins of the source group of the digital twins.

14. A method, comprising:
receiving, by a system comprising a processor, from a user equipment associated with a first user identity, a request to determine a matching subgroup of any digital twins that match, within specified matching criteria, digital twin data associated with the first user identity; and
in response to the request:
accessing a first digital twin dataset comprising first data describing first conditions related to the first user identity;
accessing respective second digital twin datasets describing respective second conditions related to respective second user identities of a group not comprising the first user identity;
determining the matching subgroup, comprising evaluating, based on the specified matching criteria, the first digital twin dataset associated with the first user identity with respect to the respective second digital twin datasets; and
communicating information related to the matching subgroup to the user equipment.

15. The method of claim 14, wherein the communicating of the information related to the matching subgroup comprises establishing a virtual anonymous communication between the user identity and the matching subgroup.

16. The method of claim 14, wherein the determining of the matching subgroup comprises evaluating at least one of: first physical data, first chemical data, first emotional data or first environmental data of the first digital twin dataset with corresponding second physical data, second chemical data, second emotional data or second environmental data of the second digital twin datasets.

17. A non-transitory machine-readable medium, comprising executable instructions that, when executed by a processor, facilitate performance of operations, the operations comprising:
collecting respective digital twin datasets from respective users of a group;
matching, based on specified matching criteria, a first digital twin dataset of a first user of the group with respect to the respective digital twin datasets to determine a second digital twin dataset of a second user of the group that satisfies the specified matching criteria; and
outputting data of the second digital twin dataset.

18. The non-transitory machine-readable medium of claim 17, wherein the collecting of the respective digital twin datasets from the respective users of the group comprises collecting respective time data for the respective digital twin datasets.

19. The non-transitory machine-readable medium of claim 17, wherein the operations further comprise establishing a virtual anonymous communication between the first user and the second user.

20. The non-transitory machine-readable medium of claim 17, wherein the matching comprises comparing at least one of: first physical data of the first digital twin dataset with second physical data of the second digital twin dataset, first chemical data of the first digital twin dataset with second chemical data of the second digital twin dataset, first emotional data of the first digital twin dataset with second emotional data of the second digital twin dataset, or first environmental data of the first digital twin dataset with second environmental data of the second digital twin dataset.
